(19) United States
(12) Patent Application Publication
(10) Pub. No.: US 2007/0185067 A1
(43) Pub. Date: Aug. 9, 2007

(54) Ciclesonide and glycopyrronium combination

(75) Inventors: Rene Roscher, Konstanz (DE); Christoph Karl, Purkersdorf (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/589,871

(22) PCT Filed: Feb. 25, 2005
(86) PCT No.: PCT/EP05/50799

§ 371 Date: Aug. 18, 2006

(30) Foreign Application Priority Data
Feb. 27, 2004 (EP) 04004473.7

(51) Int. Cl. A61K 31/58

(52) U.S. Cl. 514/174

(57) ABSTRACT

The invention relates to pharmaceutical formulations containing combinations of ciclesonide and a pharmaceutically acceptable salt of glycopyrronium and the use of such pharmaceutical compositions in medicine, in particular in the prophylaxis and treatment of respiratory disease.

### FIELD OF THE INVENTION

This invention relates to the combination of ciclesonide with glycopyrronium, in particular to pharmaceutical formulations containing combinations of ciclesonide and glycopyrronium and the use of such pharmaceutical compositions in medicine, in particular in the prophylaxis and treatment of respiratory disease.

### BACKGROUND

International patent application WO 02/36106 relates to novel medicament compositions based on anticholinergics and corticosteroids. WO 02147668 relates to novel medicament compositions based on anticholinesterase drugs and ciclesonide, to methods for the production thereof, and to their use in treating respiratory tract diseases

WO 01/76575 is related to a pharmaceutical composition for pulmonary delivery, which comprises glycopyrrolate in a controlled release formulation, wherein, on administration, the glycopyrrolate exerts its pharmacological effect over a period greater than 12 hours.

WO 00/69468 is related to novel medicament compositions, based on anticholinergic compounds and beta mimetics, which are effective on a long-term basis. The invention also relates to a method for producing the same and to their use in the treatment of diseases of the respiratory tract.

GB 247680 discloses pregna-1,4-diene-3,20-dione-16-17-acetal-21 esters and their use in the treatment of inflammatory conditions. The compounds have the general structure: 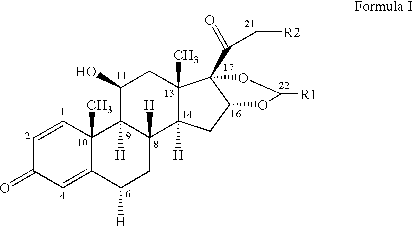
wherein R1 is 2-propyl, 1-butyl, 2-butyl, cyclohexyl or phenyl; and R2 is acetyl or isobutanoyl. Ciclesonide is the INN for a compound of formula I in which R1 is cyclohexyl and R2 is isobutanoyl with the chemical name [11β,16α(R)]-16,17-[(Cyclohexylmethylen)bis(oxy)]-11-hydroxy-21-(2-methyl-1-oxo-prop-oxy)pregna-1,4-dien-3,20-dion.

This compound has undergone evaluation as an antiasthmatic and pharmacokinetic studies show that it will be useful in an inhaler formulation. Ciclesonide is only moderately absorbed after oral administration and has low systemic activity. Concentration of the drug in the lungs is high and metabolism by liver oxidases is very high, giving the drug a low plasma half-life. Systemic activity of ciclesonide is three times lower than that of budesonide, but anti-inflammatory activity is higher for the former.

Glycopyrrolate {3-[(Cyclopentyl-hydroxyphenylacetyl)oxy]-1,1-dimethylpyrrolidinium bromide} is an anticholinergic drug, has been described for use in the treatment of incontinence (Levin et al, J. Urol., 128:396-398 (1982); Cooke et al., S. Afr. Med. J., 63:3 (1983); R. K. Mirakhur and J. W. Dundee, Anaesthesia, 38:1195-1204 (1983)). Glycopyrrolate has two centers of asymmetry (chiral centers), and can exist in four stereoisometric forms, i.e., two enantiomeric pairs of diastereomers. The two diastereomer pairs have been separated from one another (see, e.g., Demian et al, J. Liq. Chromatog., 13:779-787 (1990)). Commercially available formulations of glycopyrrolate (e.g., Robinul®, a product of A. H. Robins) contain both the (R,S)-glycopyrrolate and (S,R)-glycopyrrolate enantiomers.

U.S. Pat. No. 6,204,285 discloses methods and compositions for treating urinary incontinence using enantiomerically enriched (R,R)-glycopyrrolate, WO 98/00132 discloses methods and compositions for treating urinary incontinence using enantiomerically enriched (R,S)-glycopyrrolate and WO 98/00133 discloses methods and compositions for treating urinary incontinence using enantiomerically enriched (S,S)-glycopyrrolate. WO 98/021183 discloses enantiomerically pure pharmaceutically suitable salt of glycopyrronium [S,S-, S,R, R,S- and R,R-forms] and the use in the treatment of spasms of the smooth musculature of the gastrointestinal tract and for treating obstructive respiratory disorders.

### SUMMARY OF THE INVENTION

It has now been surprisingly found that by combined administration of a pharmaceutical acceptable salt of glycopyrronium and ciclesonide significant unexpected therapeutic benefit, particularly a synergistic therapeutic benefit, in the treatment of inflammatory or obstructive airways diseases can be obtained (e.g. higher FEV1/FVC as compared to monotherapies, dose reduction as compared to respective mono compounds).

Furthermore, using the compositions of the invention, pharmaceutical compositions, which have a rapid onset and a long duration of action may be prepared. In particular the combination therapy according to the inventions permits the establishment of a twice daily, in particular once daily dosing regimen with consequent substantial benefits in, for example the treatment of obstructive or inflammatory airways diseases (e.g. higher patient compliance, less side effects)

Thus in one aspect the present invention relates to a pharmaceutical formulation comprising a pharmaceutical acceptable salt of glycopyrronium, solvent or physiologically functional derivative thereof in combination with ciclesonide, a pharmaceutically acceptable salt, solvent or physiologically functional derivative thereof.

Ciclesonide (hereinafter also referred to as active ingredient) is the INN for a compound with the chemical name [11β,16α(R)]-16,17-[(Cyclohexylmethylen)bis(oxy)]-11-hydroxy-21-(2-methyl-1-oxo-prop-oxy)pregna-1,4-dien-3,20-dion. Ciclesonide and its preparation are disclosed in DE 4129535. Ciclesonide as used herein also includes, pharmaceutically acceptable salts of diclesonide, epimers of ciclesonide (e.g. [11β,16α(S)]-16,17-[(Cyclohexylmethylen)bis(oxy)]-11-hydroxy-21-(2-methyl-1-oxo-propoxy)-pregna-1,4-dien-3,20-dion) in any mixing ratio with ciclesonide, solvates of ciclesonide, physiologically functional derivatives of ciclesonide or solvates thereof. By the term “physiologically functional derivative” is meant a chemical derivative of ciclesonide having the same physiological function as cidesonide, for example, by being convertible in the body thereto or by being an active metabolite of ciclesonide. Physiological functional derivatives of ciclesonide which may be mentioned in connection with the invention are for example the 21-hydroxy derivative of ciclesonide with the chemical name 16α,17-(22R,S)-Cyclohexylmethylendioxy-11β,21-dihydroxypregna-1,4-dien-3,20-dion, in particular 16α,17-(22R)-Cyclohexylmethylendioxy-11β,21-dihydroxypregna-1,4-dien-3,20-dion. This compound and its preparation are disclosed in WO 9422899.

Pharmaceutical acceptable salt of glycopyrronium in connection with the invention refers to pharmacologically acceptable salts normally used in pharmaceutical technology. Pharmacologically acceptable salts which may be mentioned in connection with glycoprronium are the bromide, chloride, phosphate, nitrate, sulfate, citrate, fumarate, propionate, tartrate, iodide, benzoate, methansulfonate or tosylate salt. In a preferred embodiment of the invention the salt is the bromide salt.

Pharmaceutical acceptable salt of glycopyrronium in connection with the invention refers to the racemic forms [S,S-, S,R, R,S- and R,R-forms] of the pharmaceutical acceptable salt of glycopyrronium in any mixing ratio and preferably to the enantiomerically enriched S,S-, S,R, R,S- and R,R-forms of the pharmaceutical acceptable salt of glycopyrronium (i.e. pharmaceutically acceptable salt of (3S,2′S)-3-[(cyclopentylhydroxyphenylacetyl)oxy]-1,1-dimethylpyrrolidinium, pharmaceutically acceptable salt of (3S,2′R)-3-[(cyclopentylhydroxyphenylacetyl)oxy]-1,1-dimethylpyrrolidinium, pharmaceutically acceptable salt of (3R,2′S)-3-[(cyclopentylhydroxyphenylacetyl)oxy]-1,1-dimethylpyrrolidinium and pharmaceutically acceptable salt of (3R,2′R)-3-[(cyclopentylhydroxyphenylacetyl)oxy]-1,1-dimethylpyrroli-dinium). In a preferred embodiment of the invention the enantiomerically enriched form of the pharmaceutical acceptable salt of glycopyrronium is the R,R-form (i.e. (3R,2′R)-3-[(cyclopentylhydroxyphenylacetyl)oxy]-1,1-dimethylpyrrolidinium). Enantiomerically enriched in connection with the invention refers to a pharmaceutical acceptable salt of glycopyrronium with an enantiomeric purity of 90% minimum enantiomeric excess (ee), preferably 95% ee, more preferably more than 98% ee, and in particular preferably more than 99.5% ee. In a preferred embodiment of the invention the pharmaceutical acceptable salt of glycopyrronium in connection refers to an pharmaceutical acceptable salt of (3R,2′R)-3-[(cyclopentylhydroxyphenylacetyl)oxy]-1,1-dimethylpyrrolidinium) which substantially does not contain glycopyrronium in the S,S-, S,R- and/or R,S-forms. In a further preferred embodiment of invention the pharmaceutical acceptable salt of glycopyrronium refers to (3R,2′R)-3-[(cyclopentyl-hydroxyphenylacetyl)oxy]-1,1-dimethylpyrrolidinium) bromide, preferably enantiomerically enriched with an minimum ee of 99.5%.

The pharmaceutically acceptable salts of glycopyrronium may be prepared as disclosed in U.S. Pat. No. 6,204,285 WO 98/00132, WO 98/00133,and WO 98/021183.

It will be appreciated that the compounds of the combination may be administered simultaneously, either in the same pharmaceutical formulation (hereinafter also referred to as fixed combination) or in different pharmaceutical formulations (hereinafter also referred to as free combination) or sequentially in any order. If there is sequential administration, the delay in administering the second compound should not be such as to lose the beneficial therapeutic effect of the combination. As an example, both drugs may be provided separately as oral formulations, or one may be an oral preparation and the other an inhalant, or both may be provided in a form suitable for inhalation. Administration may be at the same time. Or they may be administered either close in time or remotely, such as where one drug is administered in the morning and the second drug is administered in the evening.

As mentioned above, both the pharmaceutical acceptable salt of glycopyrronium and ciclesonide and their pharmaceutically acceptable salts, solvates, and physiologically functional derivatives have been described for use in the treatment of respiratory diseases. Therefore, formulations of pharmaceutical acceptable salt of glycopyrronium and ciclesonide, pharmaceutically acceptable salts, solvates, and physiologically functional derivatives have use in the prophylaxis and treatment of clinical conditions for which a corticosteroid and/or an anticholinergic compound is indicated. Such conditions include diseases associated with reversible or irreversible or partially reversible airways obstruction such as asthma, nocturnal asthma, exercise-induced asthma, chronic obstructive pulmonary diseases (COPD) (e.g. chronic and wheezy bronchitis, emphysema, shortness of breath), respiratory tract infection and upper respiratory tract disease (e. g. rhinitis, such as allergic and seasonal rhinitis). The combination may be administered prophylactically or after onset of symptoms.

Accordingly, the present invention also provides a method for the prophylaxis or treatment of a clinical condition in a mammal, such as a human, for which a corticosteroid and/or an anticholinergic compound is/are indicated, which comprises administration of a therapeutically effective amount of a pharmaceutical formulation comprising ciclesonide or a pharmaceutical acceptable salt, solvate, or physiologically functional derivative thereof and a pharmaceutical acceptable salt of glycopyrronium, a solvate, or physiologically functional derivative thereof, and a pharmaceutical acceptable carrier and/or one or more excipients. In a preferred aspect, there is provided such a method, which comprises administration of a therapeutically effective amount of a combination comprising ciclesonide and pharmaceutical acceptable salt of glycopyrronium, and a pharmaceutical acceptable carrier and/or one or more excipients. In particular, the present invention provides such a method for the prophylaxis or treatment of a disease associated with reversible airways obstruction such as asthma, chronic obstructive pulmonary disease (COPD), respiratory tract infection or upper respiratory tract disease.

The amount of ciclesonide or a pharmaceutical acceptable salt, solvate or physiologically functional derivative thereof and pharmaceutical acceptable salt of glycopyrronium which is required to achieve a therapeutic effect will, of course, vary with the particular compound, the route of administration, the subject under treatment, and the particular disorder or disease being treated.

As a monotherapy, ciclesonide is generally administered to adult humans by inhalation at a daily dose of from 0,05 to 2 mg, which can be administered in one or several doses.

The dosage of the pharmaceutically acceptable salt of glycopyrronium is in the order of magnitude customary for glycopyrronium for the treatment of respiratory diseases for example in the range from 0.1 to 1000 μg.

It is preferred in connection with the present invention to have a twice daily and particularly preferred to have a once daily dosing regimen.

Suitably, the pharmaceutical formulations for inhalation according to the invention comprise the active ingredients in amounts such that in case of administration by inhalation from inhalers each actuation provides a therapeutically effective dose, for example, a dose of ciclesonide of 10 μg to 800 μg, 25 μg to 400 μg, preferably 50 μg to 200 μg (e.g. 100 μg) and a dose of pharmaceutical acceptable salt of glycopyrronium in a range of 0.1 to 1000 μg glycopyrronium bromide, preferably 30 μg, 60 μg and 120 μg. It is particularly preferred that each actuation provide a dose therapeutically effective for a twice daily dosing regiment or more particularly preferred for a once daily dosing regimen.

Suitably, the pharmaceutical formulations for inhalation according to the invention provide therapeutically effective doses that permit the establishment of a twice daily (bis in diem —b.i.d) dosing regimen and in particular a once daily dosing regimen.

The formulations include those suitable for oral, parenteral (including subcutaneous, intradermal, intramuscular, intravenous and intraaarticular, intranasal, inhalation (including fine particle dusts or mists which may be generated by means of various types of metered dose pressurised aerosols, nebulisers, liquid-based inhalers equipped with appropriate aerolization technologies/apparatus or insuflators), rectal and topical (including dermal, buccal, sublingual and intraocular administration) although the most suitable route may depend upon for example the condition and disorder of the recipiant. The formulations may conveniently be presented in unit dosage form and may be prepared by any of the methods well known in the art of pharmacy. All methods include the step of bringing the active ingredients into association with the carrier, which constitutes one or more accessory ingredients/excipients. In general the formulations are prepared by uniformly and intimately bringing into association the active ingredients with liquid carriers or finely divided solid carriers or both and then, if necessary, shaping the product into the desired formulation.

In a preferred embodiment of the invention the pharmaceutical acceptable salt of glycopyrronium and ciclesonide are provided in form suitable for inhalation. Both active ingredients may be provided in separate dosage forms (free combination) and preferably in a fixed combination.

Formulations for inhalation include powder compositions, which will preferably contain lactose, and spray compositions which may be formulated, for example, as aqueous solutions or suspensions or as aerosols delivered from pressurised packs, with the use of a suitable propellant, e. g. 1,1,1,2-terafluorethane, 1,1,1,2,3,3,3-heptafluoropropane, carbon dioxide or other suitable gas. A class of propellants, which are believed to have minimal ozone-depleting effects in comparison to conventional chlorofluorocarbons comprise hydrofluorocarbons and a number of medicinal aerosol formulations using such propellant systems are disclosed in, for example, EP 0372777, W091/04011, W091/11173, W091/11495, W091/14422, W093/11743, and EP-0553298. These applications are all concerned with the preparation of pressurised aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents or wetting agents (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example WO02/30394) and amino acids and vehicles such as cromoglicic acid and/or nedocromil which are contained at concentrations, which are not therapeutically and prophylactically active (see WO00/07567). For suspension aerosols, the active ingredients should be micronised so as to permit inhalation of substantially all of the active ingredients into the lungs upon administration of the aerosol formulation, thus the active ingredients will have a mean particle size of less than 100 microns, desirably less than 20 microns, and preferably in the range 0.7 to 10 microns, for example, 1 to 5 microns. A suitable formulation for ciclesonide based on hydrofluorocarbon propellants is for example known from WO 98/52542. WO 98/52542 is related to pharmaceutical compositions comprising a therapeutically effective amount of ciclesonide or a related compound and a hydrofluorocarbon propellant, preferably selected from 1,1,1,2-tetrafluoroethane, 1,1,1,2,3,3,3-heptafluoropropane and a mixture thereof, and cosolvent, preferably ethanol, in an amount effective to solubilize ciclesonide and optionally a surfactant.

Canisters generally comprise a container capable of withstanding the vapour pressure of the propellant, such as plastic or plastic-coated glass bottle or a metal can, for example an aluminium can which may optionally be anodised, lacquer-coated and/or plastic-coated, which container is closed with a metering valve. Canisters may be coated with a fluorocarbon polymer as described in WO 96132150, for example, a co-polymer of polyethersulphone (PES) and polytetrafluoroethylene (PTFE). Another polymer for coating that may be contemplated is FEP (fluorinated ethylene propylene).

The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in W092/11190 and valves containing EPDM rubber as described in W095/02650 may be suitable. Suitable valves are commercially available from manufacturers well known in the aerosol industry, for example, from Valois, France (eg. DF10, DF30, DF60), Bespak pic, UK (eg. BK300, BK356, BK357) and 3M-Neotechnic Ltd, UK (eg. Spraymiser).

Valve seals, especially the gasket seal and also the seals around the metering chamber, can be manufactured of a material, which is inert to and resists extraction into the contents of the formulation, especially when the contents include ethanol.

Valve materials, especially the material of manufacture of the metering chamber, can be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT.

Materials of manufacture of the metering chamber and/or the valve stem may desirably be fluorinated, partially fluorinated or impregnated with fluorine containing substances in order to resist drug deposition.

Valves, which are entirely or substantially composed of metal components (eg Spraymiser, 3M-Neotechnic), are especially preferred for use according to the invention.

Intranasal sprays or nasal drops may be formulated with aqueous or non-aqueous vehicles with or without the addition of agents such as thickening agents, buffer salts or acid or alkali to adjust the pH, isotonicity adjusting agents, preservatives or anti-oxidants.

In another embodiment of the invention the pharmaceutical formulation comprising the pharmaceutical acceptable salt of glycopyrronium in combination with ciclesonide is a dry powder, i.e. ciclesonide and the pharmaceutically acceptable salts of glycopyrronium are present in a dry powder comprising finely divided pharmaceutical acceptable salt of glycopyrronium and ciclesonide optionally together with a finely divided pharmaceutically acceptable carrier, which is preferably present and may be one or more materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder may be in capsules of gelatine or plastic, or in blisters, for use in a dry powder inhalation device, preferably in dosage units of the mixture of pharmaceutical acceptable salt of glycopyrronium and ciclesonide together with the carrier in amounts to bring the total weight of powder in each capsule to from 5 mg to 50 mg. Alternatively the dry powder may be contained in a reservoir of a multi-dose dry powder inhalation device. Capsules and cartridges of for example gelatin, or blisters of for example laminated aluminium foil, for use in an inhaler or insulator may be formulated containing a powder mix of the active ingredients and a suitable powder base such as lactose or starch, preferably lactose. In this aspect, the active ingredients are suitably micronised so as to permit inhalation of substantially all of the active ingredients into the lungs upon administration of the dry powder formulation, thus the active ingredients will have a particle size of less than 100 μm, desirably less than 20 μm, and preferably in the range 1 to 10 μm. The solid carrier, where present, generally has a maximum particle diameter of 300 μm, preferably 200 μm, and conveniently has a mean particle diameter of 40 to 100 μm, preferably 50 to 75 μm. The particle size of the active ingredients and that of a solid carrier where present in dry powder compositions, can be reduced to the desired level by conventional methods, for example by grinding in an air-jet mill, ball mill or vibrator mill, microprecipitation, spray drying, lyophilisation or recrystallisation from supercritical media.

Where the inhalable form of the composition of the invention is the finely divided particulate form, the inhalation device may be, for example a dry powder inhalation device adapted to deliver dry powder from a capsule or blister containing a dosage unit of the dry powder or a multi-dose dry powder inhalation device. Such dry powder inhalation devices are known in the art Examples which may be mentioned are Cyclohaler®), Diskhaler® Rotadisk®, Turbohaler®), Novolizer® or the dry powder inhalation devices disclosed EP 0 505 321, EP 467028, EP 650410, EP 691865 or EP 725725 (Ultrahaler®).

Formulations for inhalation by nebulization may be formulated with an aqueous vehicle with the addition of agents such as alcohols, acid or alkali, buffer salts, isotonicity adjusting agents or antimicrobials. They may be sterilised by filtration or heating in an autoclave. Suitable technologies for this type of administration are known in the art. As an example the Mystic® technology is to be mentioned (see for example U.S. Pat. No. 6,397,838, U.S. Pat. No. 6,454,193 and U.S. Pat. No. 6,302,331) as well as Respimat (Boehringer patents), e-flow (Pari patents).

Preferred unit dosage formulations are those containing a pharmaceutical effective dose, as hereinbefore recited, or an appropriate fraction thereof, of the active ingredient. Thus, in the case of formulations designed for delivery by metered dose pressurised aerosols, one actuation of the aerosol may deliver half of the therapeutical effective amount such that two actuations are necessary to deliver the therapeutically effective dose.

It should be understood that in addition to the ingredients particularly mentioned above, the formulations of this invention may include other agents conventional in the art having regard to the type of formulation in question. Furthermore, the claimed formulations include bioequivalents as defined by the US Food and Drugs Agency.

The invention will now be illustrated by the following examples without restricting it.

Fixed Combinations

### EXAMPLE 1

### Powder Inhaler (Mono Dose System Based on Inhalation Capsule)

240 mg micronised R,R-glycopyrronium bromide, 800 mg micronised ciclesonide and 28.8 g lactose monhydrate (Ph. Eur. 4) are mixed in a turbula mixer in two steps. The blend is screened (0.71 mm sieve) and transferred to the container of a planetary mixer. After adding with additional 70.0 g lactose monohydrate and mixing, 25 mg of the blend are filled into capsules of size 3, which can be administered with a powder inhaler. One capsule contains 60 μg R,R-glycopyrronium bromide and 200 μg of ciclesonide.

### EXAMPLE 2

### Powder Inhaler (Multi Dose System)

1000 g lactose monohydrate (Ph. Eur. 4) is screened by a sieve-mill. R,R-glycopyrronium bromide micronised (screened; 0.5 mm sieve) and 147.5 g of deagglomerated lactose monohydrate are blended in a turbula mixer. 195 g of deagglomerated lactose monohydrate are filled in a high shear mixer and 5.0 g of ciclesonide micronised (screened, 0.5 mm sieve) are added to form a blend. The R,R-glycopyrronium bromide lactose pre-blend is screened (0.5 mm sieve), added to the container of a high shear mixer and mixed with the cidesonide lactose blend. Subsequently 650 g of deagglomerated lactose monohydrate are added and mixed. 1.5 g of the blend are filled in the reservoir of a multi dose powder inhaler. After fully assembling, the powder inhaler is wrapped into a protective foil to achieve moisture protection. Such powder inhaler will contain 60 single doses (20 mg powder) each containing 100 μg ciclesonide and 30 μg R,R-glycopyrronium bromide per dose.

### EXAMPLE 3

### Powder Inhaler (Multi Dose System)

R,R-glycopyrronium bromide and 14.7 g lactose monohydrate (Ph. Eur. 4) are screened (0.5 mm sieve) and mixed in a turbula mixer. The blend obtained is screened (0.5 mm sieve) and together with 10.67 g micronised ciclesonide (screened; mesh 0.5 mm) and 169.3 g lactose monohydrate (Ph. Eur. 4) filled in a steel batching vessel and blended in a turbula mixer. 1.2 g of the blend thus obtained is filled in the powder reservoir of a powder inhaler. After fully assembling the powder inhaler is wrapped in a protective foil to achieve protection from moisture. Such powder inhaler may contain at least 120 single doses (7.5 mg powder) each having 400 μg ciclesonide and 120 μg R,R-glycopyrronium bromide perdose.

B. Free Combinations

### EXAMPLE 1

R,R-glycopyrronium bromide is provided as DPI formulation and ciclesonide is provided as pharmaceutical product comprising an aerosol vial equipped with a dispensing valve and containing the following formulation:

| | | |
|---|---|---|
| Ciclesonide | 1.000 | mg/ml |
| Ethanol | 94.800 | mg/ml |
| P134a | 1090.200 | mg/ml |

Although the invention has been described in terms of preferred formulations and ingredients, it will be understood that these are not intended to be limiting. To the contrary, those skilled in the art will understand that various optional ingredients may be included, such as flavouring agents, preservatives, additional active ingredients, and the like, while still embodying the present invention.

1. A pharmaceutical formulation comprising a pharmaceutical acceptable salt of glycopyrronium, a solvate or physiologically functional derivative thereof in combination with ciclesonide, a pharmaceutically acceptable salt, solvate or physiologically functional derivative thereof and a pharmaceutically acceptable carrier and/or one or more excipients.

2. The formulation according to claim 1, wherein the pharmaceutical acceptable salt of glycopyrronium and ciclesonide are contained in the same pharmaceutical formulation (fixed combination).

3. The formulation according to claim 1, wherein the pharmaceutical acceptable salt of glycopyrronium and ciclesonide are contained in different pharmaceutical formulations (free combination).

4. The formulation according to claim 1, comprising a compound selected from the group consisting of [11β,16α(R)]-16,17-[(Cyclohexylmethylen)bis(oxy)]-11-hydroxy-21-(2 -methyl-1-oxopropoxy)pregna-1,4-dien-3,20-dion, [11β,16α(S)]-16,17-[(Cyclohexylmethylen)bis(oxy)]-11-hydroxy-21-(2-methyl-1-oxoprop-oxy)pregna-1,4-dien3,20-dion,[11β,16α(R,S)]-16,17-[(Cyclohexyl-methylen)bis(oxy)]-11-hydroxy-21-(2-methyl-1-oxoprop-oxy)pregna-1,4-dien3,20-dion,16α,17-(22R)-Cyclohexylmethylendioxy-11β,21-dihydroxy-pregna-1,4-dien-3,20-dion,16α,17-(22S)-Cyclohexylmethylendioxy-11β,21-dihydroxy-pregna-1,4-dien-3,20-dion and 16α,17-(22R,S)-Cyclohexylmethylendioxy-11β,21-dihydroxy-pregna-1,4-dien-3,20-dion.

5. The formulation according to claim 1, wherein the pharmaceutical acceptable salt of glycopyrronium is selected from the group consisting of racemic forms [S,S-, S,R, R,S- and R,R-forms] of the pharmaceutical acceptable salt of glycopyrronium in any mixing ratio and enantiomerically enriched S,S-, S,R, R,S- and R,R-forms of the pharmaceutical acceptable salt of glycopyrronium.

6. The formulation according to claim 5, wherein the enantiomerically enriched form of the pharmaceutical acceptable salt of glycopyrronium is the R,R-form (i.e. (3R,2′R)-3-[(cyclopentylhydroxyphenylacetyl)oxy]-1,1-dimethylpyrrolidinium).

7. The formulation according to claim 6, wherein the R,R-form has an enantiomeric purity of 90% minimum enantiomeric excess (ee).

8. The formulation according to claim 1, wherein the pharmaceutical acceptable salt of glycopyrronium is (3R,2′R)-3-[(cyclopentylhydroxyphenylacetyl)oxy]-1,1-dimethylpyrrolidinium bromide, which substantially does not contain glycopyrronium in the S,S-, S,R- and/or R,S- forms.

9. The formulation according to claim 1, comprising pharmaceutical acceptable salt of glycopyrronium and ciclesonide in an amount and ratio to be effective for a twice or once daily treatment of a clinical condition in a mammal, for which a corticosteroid and/or an anticholinergic agent is indicated.

10. The formulation according to claim 1, which is suitable for administration by inhalation.

11. The formulation according to claim 1, which is suitable for nasal administration.

12. The formulation according to claim 1, which is a dry powder and the carrier is a saccharide.

13. The formulation according to claim 12, wherein the carrier is lactose monohydrate.

14. A method of treatment of a clinical condition in a mammal, for which a corticosteroid and/or an anticholinergic agent is indicated, which comprises administration of a therapeutically effective amount of a pharmaceutical formulation comprising ciclesonide or a pharmaceutical acceptable salt, solvate, or physiologically functional derivative thereof in combination with a pharmaceutical acceptable salt of glycopyrronium, a solvate, or physiologically functional derivative thereof, and a pharmaceutical acceptable carrier and/or one or more excipients.

15. The method according to claim 14, wherein the clinical condition is selected from the group consisting of asthma, nocturnal asthma, exercise-induced asthma, chronic obstructive pulmonary diseases (COPD), chronic bronchitis, wheezy bronchitis, emphysema, shortness of breath, respiratory tract infection, upper respiratory tract disease, rhinitis, allergic rhinitis and seasonal rhinitis.

16. The method according to claim 15, which comprises a twice daily dosage regimen.

17. The method according to claim 15, which comprises a once daily dosage regimen.

18. The method according to claim 15, which comprises administration of a combination of a pharmaceutical acceptable salt of glycopyrronium and ciclesonide in the same administration form by inhalation from an inhaler and wherein each actuation provides a dose therapeutically effective for a twice daily dosing regiment or for a once daily dosing regiment.

19. A dry powder inhalation product comprising a pharmaceutical composition according to claim 13.
